(19) United States
(12) Patent Application Publication
(10) Pub. No.: US 2011/0120460 A1
(43) Pub. Date: May 26, 2011

(54) SYSTEM FOR RESPIRATING PATIENTS

(75) Inventors: Oliver WALLNEWITZ, Luebeck (DE); Ludger TAPPEHORN, Luebeck (DE); Kirill KOULECHOV, Niendorf (DE)

(73) Assignee: Drager Medical GmbH, Lubeck (DE)

(21) Appl. No.: 12/898,940

(22) Filed: Oct. 6, 2010

(30) Foreign Application Priority Data
Nov. 25, 2009 (EP) 09 176 992.7

(51) Int. Cl. A61M 16/06; A62B 9/04

(52) U.S. Cl. 128/202.27

(57) ABSTRACT

A respiration system (1) has a mask (2), a connection device (5) formed on the mask (2) for connecting the mask (2) with a flexible air tube, a head support (7) with a support surface (17) for being placed on the forehead of a patient and a strap system (10) for fastening the mask (2) on the head of a patient. A fastening device (14) for the strap system (10) is able to be used with a small effort both as a breathing mask (3) and as an anesthesia mask (4). This is accomplished by the head support (7) and fastening device (14), especially all fastening features (14), being able to be fastened detach ably to the mask (2), so that the system (1) can be used with the mask (2) only without head support (7) and without fastening device (14) as an anesthesia mask (4) and the mask (2) with head support (7) and with fastening device (14) can be used as a breathing mask (3).

### CROSS REFERENCE TO RELATED APPLICATIONS

This application claims the benefit of priority under 35 U.S.C. §119 of European Patent Application EP 09 176 992.7 filed Nov. 25, 2009, the entire contents of which are incorporated herein by reference.

### FIELD OF THE INVENTION

The present invention pertains to a system for respirating (also known as ventilating) patients.

### BACKGROUND OF THE INVENTION

Respirators (also known as respirators) are used for the noninvasive respiration of patients. The respirators comprise a breathing mask, which is placed on the patient's face area and covers, in general, both the nose and mouth, so that the patient can be supplied with artificial breathing air over a rather long period of time during breathing through the nose and mouth. Respirators with a breathing mask comprise, moreover, in general, a head support and a strap system.

In addition, respirators may also have anesthesia masks. Anesthesia masks are likewise placed on the patient's face area, so that the nose and mouth are covered. Respirators with anesthesia masks are placed on the patient's face area only briefly and therefore do not comprise a strap system for durable fastening to the patient's head like breathing masks, and also no head support. Respirators with breathing masks must therefore be disadvantageously stocked for the artificial respiration of patients and respirators with anesthesia masks must be stocked for anesthesia. Thus, this requires a great expense for stocking and manufacturing the two different respirators.

A respirator is known from US 2003/0034034 A1. The respirator with a breathing mask comprises a head support, so that the respirator can be used only as a breathing mask but not as an anesthesia mask.

### SUMMARY OF THE INVENTION

The object of the present invention is therefore to make available a system for respirating patients, which can be used with little effort both as a breathing mask and as an anesthesia mask. The system for respirating patients shall, furthermore, be able to be handled in a simple manner and reliably and have low manufacturing costs.

This object is accomplished with a system for respirating patients or a respirator, comprising a mask, a connection means formed on the mask for connecting the mask with a flexible air tube, preferably a head support with a support surface for placing it on the forehead of a patient, preferably a strap system for fastening the mask to the head of a patient, preferably fastening means for the strap system, wherein the head support and the fastening means can be detachably fastened to the mask, so that the system can be used without head support and without fastening means, preferably with the mask only, as an anesthesia mask, and the system with head support and with fastening means can be used as a breathing mask. The components of the system, namely, the mask, connection means, head support, strap system and fastening means, can thus be combined with one another, so that a breathing mask is obtained when all components are used and an anesthesia mask is obtained if only some of the components are used. The components not used are stored at a different place for later use of all components.

The head support and fastening means, especially all fastening means, can be detached from the mask. The mask can thus be used as a breathing mask if it is used with the head support and with the fastening means. A breathing mask requires a head support and fastening means because a strap system can be attached to the fastening means for durably fixing the breathing mask to the face area of a patient to be artificially respirated. Breathing masks are fastened to the patient's face area over a rather long period of time because of artificial respiration, so that a head support is necessary to prevent injuries from occurring in the area of the nose because of the breathing mask. Anesthesia masks are placed on the patient's face area for a short period of time only in order to supply the patient with gaseous anesthetic over only a short period of time. Anesthesia masks therefore require no head support and also no fastening means and also no strap system, either, which would be fixed or fastened to the fastening means. Anesthesia masks are placed, in general, only by the anesthesiologist by hand on the face area and are also held by hand on the face area for supply with gaseous anesthetic.

In another embodiment, the head support and fastening means, especially all fastening means, are designed as a separate component and/or all fastening means can be detachably fastened to the mask.

The component can be detachably fastened to the mask in an additional embodiment. The head support and all fastening means are formed on the separate component. If the system is used to respirate patients as a breathing mask, the component is fastened to the mask, so that the system for respirating patients also comprises as a result the head support and fastening means. Thus, the strap system can also be attached to the fastening means, and thus there is a system with a breathing mask. To use the system as an anesthesia mask, i.e., if the mask is used as an anesthesia mask, only the component needs to be removed from the mask because of the detachable connection with the mask, so that only the mask is present as an anesthesia mask without the head support and fastening means. This mask as an anesthesia mask can then be placed by an anesthesiologist on the patient's face area. It is thus advantageously possible to make available with the system both an anesthesia mask and a breathing mask because of the detachable fastening of the head support and the fastening means to the mask.

In one variant, the component comprises a ring. Thus, the ring represents a fixing means for fixing the component to the mask. The fixing means may also be designed, besides as a ring, as a screw or locking connection or clamping connection.

In an additional embodiment the mask comprises a connection piece, especially as a connection means for connecting the mask with the flexible air tube. The connection piece thus represents a counterfixing means, and the fixing means can be attached to the component by means of the counterfixing means on the mask. The counterfixing means formed on the mask may also be, for example, a thread or a recess for a locking connection.

In an additional embodiment, the ring of the component can be pushed over the connection piece for detachably connecting the component with the mask.

The head support and fastening means can be detachably fastened to the mask by means of a screw or locking connection in another variant. The screw or locking connection thus represents a fixing device.

The fastening means are preferably designed as at least one hook and/or at least one eyelet and/or at least one slot.

The strap system comprises a head strap and side straps in an additional embodiment.

The strap system is elastic in an additional embodiment.

The position of the support surface of the head support relative to the mask is adjustable, especially in a fixable manner.

In an additional embodiment, the head support has an air-filled support means of variable volume, so that the position of the support surface of the head support relative to the mask can be adjusted by means of a variable volume of air in the support means.

In an additional embodiment, the support means is a preferably elastic balloon and the support means preferably comprises a valve for filling and emptying the balloon. The balloon is manufactured from an elastic material and thus lies on the patient's forehead. The forces applied by the head support, i.e., the balloon, to the forehead can thus be advantageously transmitted especially uniformly to the surface of the patient's forehead. The balloon can also be filled with air and air can be emptied from it because of the valve, so that the volume is variable as a result and the head support, i.e., the support surface of the head support, relative to the mask can be adjusted and the head support can thus be adapted to different patient face shapes.

In another embodiment, the mask and/or head support and/or strap system are made at least partly and preferably completely of plastic.

The fastening means are preferably formed exclusively on the separate component.

The strap system preferably consists at least partly of a fabric, especially synthetic fibers.

An exemplary embodiment of the present invention will be described in more detail below with reference to the drawings attached. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a side view of a system for respirating patients;

FIG. 2 is a front view of a head support and fastening means as a separate component of the system according to FIG. 1; and

FIG. 3 is a side view of the component according to FIG. 2.

### DESCRIPTION OF THE PREFERRED EMBODIMENTS

Referring to the drawings in particular, systems 1 for respirating patients are used for the non-invasive artificial respiration of patients and for the anesthesia of patients. The system 1 for respirating patients comprises a mask 2 with a connection means 5 as a connection piece 6 for connecting and detachably fixing a flexible air tube, not shown. Due to its geometry, mask 2 can cover both the nose and mouth of a patient, i.e., artificially respirating both the nose and the mouth simultaneously.

Mask 2 can be used both as a breathing mask 3 for the artificial respiration of patients and as an anesthesia mask 4 for anesthesia. Anesthesia masks 4 are placed on the face area of a patient only briefly and held by the hand of an anesthesiologist while being placed. When the mask 2 of system 1 for respirating patients is used as an anesthesia mask 4, system 1 for respirating patients thus does not include any head support 7 and any strap system 10. When the mask 2 of system 1 is used as a breathing mask 3, the system 1 includes the head support 7 and strap system 10.

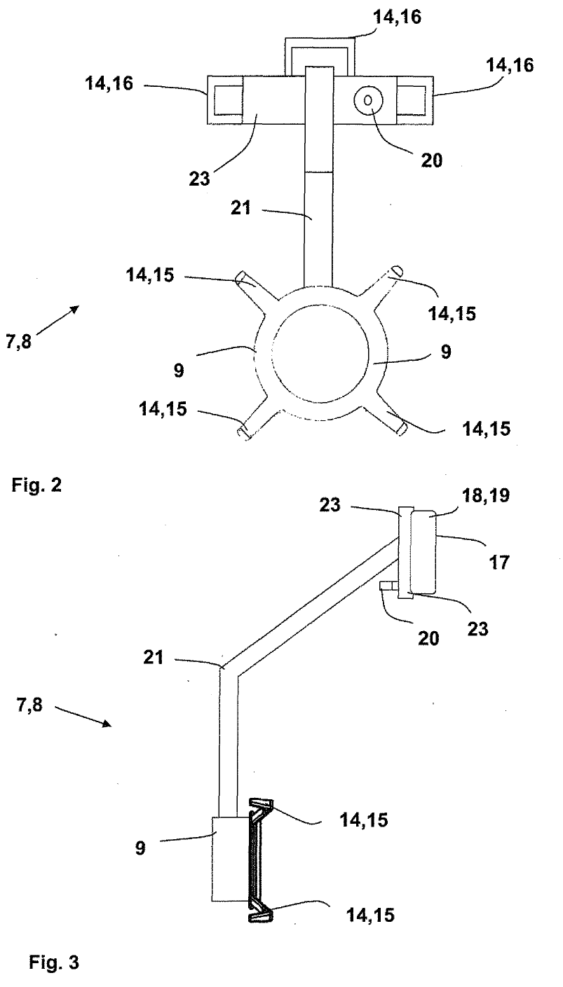

The head support 7 made essentially of plastic comprises a head support arm 21. A ring 9 with hook 15 are arranged as fastening means 14 at a lower end of the head support arm 21 (FIG. 2). A support plate 23 is arranged at the upper end of the support arm 21 (FIGS. 2 and 3). Eyelets 16 are formed as fastening means 14 on the support plate 23. In addition, a support means 18 designed as a balloon 19 is present at the support plate 23. The support means 18, i.e., balloon 19 with a support surface 17, is used to be placed on the forehead of a patient to be respirated in order to make it possible to apply forces on the forehead by means of the support surface 17. The head support 7 is provided, furthermore, with a valve 20 at the support plate 23. Air can be filled into and removed from the elastic balloon 19 made of plastic by means of valve 20, so that the volume of balloon 19 is variable as a result. The relative direction of the support surface 17 of the head support 7 relative to the mask 2 can be changed in this way and the head support 7 can thus be adapted to different patient head shapes. The design of the support means 18 as a balloon 19 makes it, furthermore, advantageously possible to transmit the forces at the support surface 17 to the forehead of the patient, so that no injuries due to strong local forces can advantageously develop as a result on the patient's forehead. Ring 9 of the head support 7 is used as a fixing means for fixing the head support 7 to the mask 2 if the mask 2 is used as a breathing mask 3. Only the ring 9 of the head support 7 is pushed for this in a simple manner onto the connection piece 6 as a connection means 5 for the flexible air tube of the mask 2 before the flexible air tube is attached to the connection piece 6. Only the ring 9 needs to be pushed off from the connection piece 6 to remove the head support 7 from mask 2.

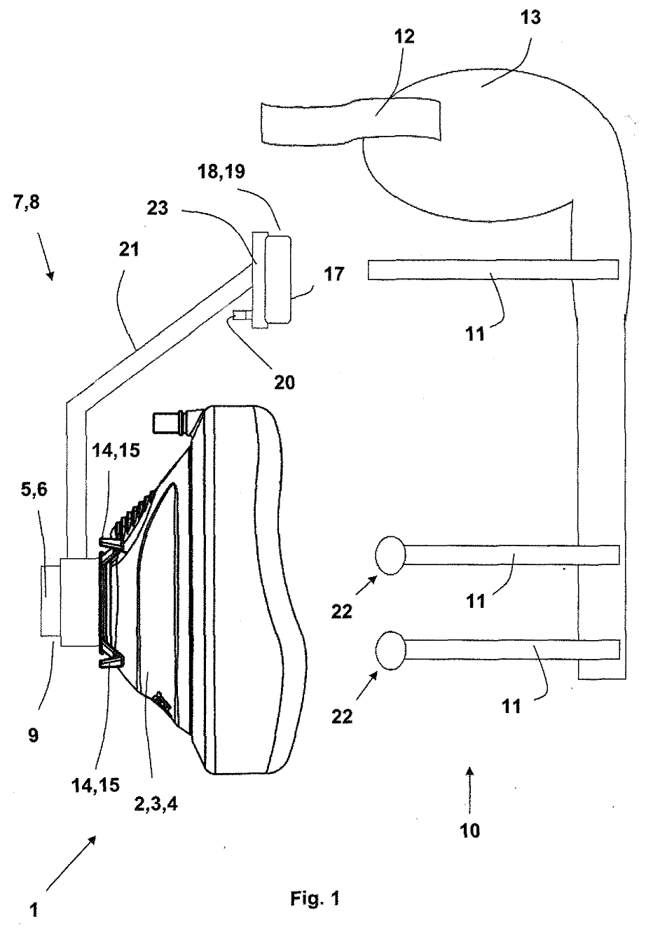

If the mask 2 of the system 1 for respirating patients is used as a breathing mask 3, the system 1 also comprises a strap system 10 (FIG. 1). Strap system 10 comprises here six side straps 11, of which only three are visible in FIG. 1 because of the perspective, as well as a head strap 12. The head strap 12 is fastened here to a large-area surface element 13 of the strap system 10. Fastening straps 22 of the side straps 10 are hung onto the hook 15 of ring 9 for durably fastening the breathing mask 3 to the surface of the face of a patient to be respirated artificially. Head strap 12 and the two upper side straps 11 have a Velcro fastener, not shown, by means of which [they] can be fastened to the rest of the side strap 11 and the rest of the head strap 12 after introducing the head strap 12 and the side straps 11 into the eyelets 16 at the support plate 23, so that the two upper side straps 11 and the head strap 12 are fastened as a result to the eyelets 16 of the support plate 23. The breathing mask 3 can thus be durably fastened to the face area of a patient. The strap system 10 is, in addition, elastic, so that the strap system 10 can adapt itself as a result to different sizes and head shapes of patients [sic—probably “different patient head sizes and shapes—Tr.Ed.].

On the whole, essential advantages are associated with the system 1 according to the present invention for respirating patients. The mask 2 of system 1 can be used both as a breathing mask 3 and as an anesthesia mask 4. It is therefore unnecessary to stock different masks 2 for a breathing mask 2 as well as an anesthesia mask 4 as part of system 1 for respirating patients, but with one mask 2, said mask can be used either both as an anesthesia mask 4 without the head support 7 and without the strap system 10 and it can be used with the head support 7 and with the strap system 10 in case of use as a breathing mask 3. The head support 7 can be pushed with ring 9 over the connection piece 6 in a simple manner, so that the mask can be converted from an anesthesia mask 4 into a breathing mask 3 in a very short time. High costs for the manufacture of different masks 2 as a breathing mask 3 and as an anesthesia mask 4 can thus be avoided, and, on the other hand, it is possible to stock, for example, in hospitals, only one mask, which can be used both as a respirating mash 3 and as an anesthesia mask 4.

While specific embodiments of the invention have been described in detail to illustrate the application of the principles of the invention, it will be understood that the invention may be embodied otherwise without departing from such principles.

### APPENDIX

### List of Reference Numbers

1 System for respirating patients
2 Mask
3 Breathing mask
4 Anesthesia mask
5 Connection means
6 Connection piece
7 Head support
8 Component
9 Ring
10 Strap system
11 Side strap
12 Head strap
13 Surface element
14 Fastening means
15 Hook
16 Eyelet
17 Support surface
18 Support means
19 Balloon
20 Valve
21 Head support arm
22 Fastening strap
23 Support plate

What is claimed is:

1. A system for respirating patients, the system comprising
a mask;
a flexible air tube;
a connection means formed on the mask for connecting the mask with the flexible air tube;
a head support with a support surface for being placed on the forehead of a patient;
a strap system for fastening the mask to the head of a patient; and
a fastening means for the strap system wherein the head support and the fastening means can be detachably fastening to the mask, so that the system is used as an anesthesia mask without head support and without the fastening means and the system can be used as a breathing mask with head support and with fastening means.

2. A system in accordance with claim 1, wherein the head support and fastening means are each a separate component and/or the fastening means comprises plural fasteners that can be detachably fastened to the mask.

3. A system in accordance with claim 2, wherein each separate component can be detachably fastened to the mask.

4. A system in accordance with claim 2, wherein said fastening means separate component comprises a ring.

5. A system in accordance with claim 4, wherein the mask comprises a connection piece forming the connection means for connecting the mask with the flexible air tube.

6. A system in accordance with claim 5, wherein the ring can be pushed onto the connection piece for detachably connecting the component with the mask.

7. A system in accordance with claim 1, wherein the head support and the fastening means are detachably fastened to the mask by means of a screw or locking connection.

8. A system in accordance with claim 1, wherein the fastening means comprises at least one hook and/or at least one eyelet and/or at least one slot.

9. A system in accordance with claim 1, wherein the strap system comprises a head strap and side straps.

10. A system in accordance with claim 1, wherein the strap system is elastic.

11. A system in accordance with claim 1, wherein the position of the support surface of the head support relative to the mask is adjustable in a fixable manner.

12. A system in accordance with claim 11, wherein the head support has an air-filled support means of variable volume, so that the position of the support surface of the head support relative to the mask is adjustable by means of a variable volume of air in the support means.

13. A system in accordance with claim 12, wherein the support means comprises an elastic balloon and the support means comprises a valve for filling and emptying the balloon.

14. A system in accordance with claim 1, wherein the mask and/or head support and/or strap system are made at least partly or completely of plastic.

15. A system in accordance with claim 1, wherein the fastening means is formed exclusively on the separate component.

16. A system for respirating patients, the system comprising
a mask;
a flexible air tube;
a connector on the mask for connecting the mask with the flexible air tube;
a head support with a support surface for engaging the forehead of a patient;
a strap system for fastening the mask to the head of a patient; and
a fastening arrangement for fastening the strap system, wherein the head support and the fastening arrangement are detachably fastenable to the mask, whereby the system provides an anesthesia mask without the head support and without the fastening arrangement and the system provides a breathing mask with the head support and with the fastening arrangement.

17. A system in accordance with claim 16, wherein the head support and the fastening arrangement are each a separate component and/or the fastening arrangement comprises plural fasteners that can be detachably fastened to the mask.

18. A system in accordance with claim 17, wherein:
the mask comprises a connection piece forming the connector for connecting the mask with the flexible air tube;
the fastening arrangement is a separate component comprising a ring; and
the ring can be pushed onto the connection piece for detachably connecting the component with the mask.

19. A system in accordance with claim 17, wherein:
the strap system comprises a head strap and side straps.
the strap system is elastic; and
the support surface of the head support relative to the mask is adjustable in a fixable manner.

20. A process for respirating patients, the process comprising:
providing a respiration system comprising a mask, a flexible air tube, a connector on the mask for connecting the mask with the flexible air tube, a head support with a support surface for engaging the forehead of a patient, a strap system for fastening the mask to the head of a patient, and a fastening arrangement for fastening the strap system;
deploying the respiration system including one of:
deploying the respiration system as an anesthesia mask without the head support fastened to the mask and without the fastening arrangement fastened to the mask; and
deploying the respiration system as a breathing mask with the head support and with the fastening arrangement wherein the head support and the fastening arrangement are detachably fastened to the mask.
